(19) United States
(12) Patent Application Publication
(10) Pub. No.: US 2025/0345434 A1
(43) Pub. Date: Nov. 13, 2025

(54) BORONATED HYDROGELS AND METHODS OF MAKING AND USING THE SAME

(71) Applicant: Boston Scientific Scimed, Inc., Maple Grove, MN (US)

(72) Inventors: Joseph Thomas Delaney, JR., Minneapolis, MN (US); Yen-Hao Hsu, Shrewsbury, MA (US)

(73) Assignee: Boston Scientific Scimed, Inc., Maple Grove, MN (US)

(21) Appl. No.: 19/202,871

(22) Filed: May 8, 2025

Related U.S. Application Data
(60) Provisional application No. 63/645,468, filed on May 10, 2024.

(51) Int. Cl. A61K 41/00; A61K 9/06

(52) U.S. Cl. CPC A61K 41/0095; A61K 9/06

(57) ABSTRACT

In some aspects, the present disclosure provides crosslinked reaction products of (a) a boronated multifunctional compound comprising a plurality of nucleophilic groups and (b) a reactive polymer comprising a plurality of electrophilic groups that react with the plurality of nucleophilic groups to form covalent linkages. In some aspects, the present disclosure pertains to a system for forming such crosslinked reaction products. In some aspects, the present disclosure pertains to methods of treatment comprising (a) applying or injecting such crosslinked reaction products or precursors thereof onto or into target tissue of a subject and (b) delivering neutron beam radiation to the target tissue, the crosslinked reaction product, or both.

### CROSS-REFERENCE TO RELATED APPLICATIONS

This application claims the benefit of U.S. Provisional Patent Application Ser. No. 63/645,468 filed on May 10, 2024, the disclosure of which is incorporated herein by reference.

### FIELD

The present disclosure relates to boronated hydrogels and to methods of making and using such hydrogels, among other aspects. The boronated hydrogels of the present disclosure are useful in biomedical applications, including boron neutron capture therapy.

### BACKGROUND

Bioresorbable hydrogels with rapid crosslinking reaction rates in vivo, known by the trade name of SpaceOAR®, have become a prominent biomaterial and obtained clinical success in creating the space between prostate and rectum, tremendously improving patient safety during the cancer therapies. SpaceOAR® is based on a multi-arm polyethylene glycol (PEG) polymer with a polyol core functionalized with succinimidyl glutarate (SG) as reactive end groups which further react with trilysine to form crosslinks. A further improvement based on this application is that some of 8-Arm PEG branches are functionalized with 2,3,5-triiiodobenzamide (TIB) groups, replacing part of the SG groups, in order to provide intrinsic radiopacity to the hydrogels themselves for CT-visibility. This hydrogel is known by the trade name of SpaceOAR Vue®. The hydrogels break down in-vivo over the course of ca. 6-9 months. The breakdown occurs primarily through the hydrolysis of the ester linkages on the glutarate groups.

Boron neutron capture therapy (BNCT) is a promising and efficient tool whereby cancers are treated by selectively concentrating boron-compounds close to or in tumor cells and then delivering neutron beam radiation for cancer therapy. Boron neutron capture therapy utilizes boronated agents to preferentially deliver boron-10 atoms to tumors. After undergoing irradiation with neutrons, boron-10 yields litihium-7 and an alpha particle. The alpha particle has a short range, therefore preferentially treats tumor tissues while sparing more distant healthy tissues. More particularly, boron neutron capture therapy is based on nuclear capture and fission that follows irradiation of nonradioactive boron-10 with low thermal neutrons which leads to the production of an alpha particle and a recoiling lithium-7 particle (¹⁰B+¹n→[¹¹B]*→⁴He₂(α)+⁷Li₃+2.38 MeV). Alpha particles are a form of high linear energy transfer (LET) particles that deposit their energy over <10 μm. For further information, see, e.g., K. Nedunchezhian. et al., Boron neutron capture therapy-a literature review, Journal of clinical and diagnostic research: JCDR, 10(12), ZE01 (2016) and T. D. Malouff et al., Boron neutron capture therapy: A review of clinical applications. Frontiers in oncology, 11, 601820 (2021).

There is a need in the biomedical arts for hydrogels that are boronated, rendering them potentially useful for boron neutron capture therapy, for methods of making and using such boronated hydrogels, and for systems for forming such boronated hydrogels.

### SUMMARY

In some aspects, the present disclosure provides a crosslinked reaction product of (a) a boronated multifunctional compound comprising a plurality of nucleophilic groups and (b) a reactive polymer that comprises a plurality of electrophilic groups that react with the plurality of nucleophilic groups to form covalent linkages.

In some embodiments, the crosslinked reaction product is a hydrogel.

In some embodiments, which can be used in conjunction with the above aspects and embodiments, the boronated multifunctional compound is a boronated polyamino compound that comprises one or more boron atoms and a plurality of amino groups. In some of these embodiments, the boronated polyamino compound comprises a polyamino moiety that comprises a plurality of amino groups linked to a boronated moiety that comprises one or more boron atoms. In some of these embodiments, the polyamino moiety is linked to the boronated moiety through a thioester group, an ester group, or an amide group.

In some embodiments, which can be used in conjunction with the above aspects and embodiments, the polyamino moiety comprises a plurality of —(CH2)x-NH2 groups where x is 1, 2, 3, 4, 5 or 6.

In some embodiments, which can be used in conjunction with any of the above the boronated polyamino compound comprises a poly (amino acid) residue.

In some embodiments, which can be used in conjunction with the above aspects and embodiments, the boronated polyamino compound comprises a trilysine residue.

In some embodiments, which can be used in conjunction with the above aspects and embodiments, the boronated moieties are selected from dioxaborolane-containing moieties and dodecaborate-containing moieties.

In some embodiments, which can be used in conjunction with the above aspects and embodiments, the boronated multifunctional compound comprises an undecahydrododecaborate group or a 4,4,5,5-tetramethyl-1,3,2-dioxaborolan-2-yl group.

In some embodiments, which can be used in conjunction with the above aspects and embodiments, the boronated polyamino compound comprises a residue of a thiol-functionalized dodecaborate-containing compound, a residue of a hydroxy-functionalized dioxaborolane-containing compound, or a residue of an amino-functionalized dioxaborolane-containing compound.

In some embodiments, which can be used in conjunction with the above aspects and embodiments, the reactive polymer is a reactive multi-arm polymer that comprises a plurality of hydrophilic polymer arms having electrophilic end groups.

In some embodiments, which can be used in conjunction with the above aspects and embodiments, the reactive polymer is a reactive multi-arm polymer that comprises three or more polymer arms linked to a core region, each of the polymer arms comprising a hydrophilic polymer segment and an electrophilic end group.

In some embodiments, which can be used in conjunction with the above aspects and embodiments, the electrophilic end group is a cyclic imide ester group.

In some embodiments, which can be used in conjunction with the above aspects and embodiments, each of the polymer arms comprises a hydrolysable ester group disposed between the hydrophilic polymer segment and the electrophilic end group.

In some embodiments, which can be used in conjunction with the above aspects and embodiments, the hydrophilic polymer segment is selected from polyalkylene oxide segments, polyester segments, polyoxazoline segments, polydioxanone segments, and polypeptide segments.

In some embodiments, which can be used in conjunction with the above aspects and embodiments, the core region comprises a polyol residue.

In some aspects, the present disclosure pertains to systems for forming a crosslinked reaction product in accordance with the above aspects and embodiments, which comprise a first composition that comprises a boronated multifunctional compound in accordance with the above aspects and embodiments and a reactive polymer in accordance with the above aspects and embodiments in a first container, a second composition that comprises an acidic buffer in a second container, and a third composition that comprise a basic buffer in a third container.

In some embodiments, the first container, the second container and the third container are independently selected from vials and syringe barrels.

In some aspects, the present disclosure pertains to methods of treatment comprising (a) applying or injecting a crosslinked reaction product in accordance with the above aspects and embodiments onto or into target tissue of a subject and (b) delivering neutron beam radiation to the target tissue, the crosslinked reaction product, or both.

The above and other aspects, embodiments, features and benefits of the present disclosure will be readily apparent from the following detailed description.

### BRIEF DESCRIPTION OF THE DRAWINGS

FIG. 1 schematically illustrates a method of making a boronated polyamino compound, in accordance with an embodiment of the present disclosure.

FIG. 2 schematically illustrates a crosslinking reaction between succinimidyl-glutarate-terminated eight-arm PEG and the boronated polyamino compound of FIG. 1, in accordance with an embodiment of the present disclosure.

FIG. 3 illustrates a delivery device, in accordance with an embodiment of the present disclosure.

FIG. 4 illustrates a delivery device, in accordance with another embodiment of the present disclosure.

### DETAILED DESCRIPTION

In some aspects, the present disclosure pertains to boronated multifunctional compounds that comprise a plurality of nucleophilic groups and one or more boron atoms. The boronated multifunctional compounds are useful, for example, as crosslinkers for reactive polymers that have a plurality of electrophilic groups, which are reactive with the nucleophilic groups of the boronated multifunctional compound.

Nucleophilic groups for use in the boronated multifunctional compounds of the present disclosure include amine groups, particularly primary amine groups, and thiol groups.

Electrophilic groups for use in the reactive polymers of the present disclosure include activated ester groups, including cyclic imide ester groups such as succinimide ester groups,

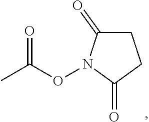

maleimide ester groups, glutarimide ester groups, diglycolimide ester groups, phthalimide ester groups, and bicyclo[2.2.1]hept-5-ene-2,3-dicarboxylic acid imide ester groups,

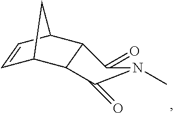

imidazole ester groups, imidazole carboxylate groups and benzotriazole ester groups, among others.

Boron has two naturally-occurring stable isotopes, ¹⁰B (19.9%) and ¹¹B (80.1%). In some embodiments, the boron isotopes in the boronated multifunctional compounds of the present are present in naturally occurring relative amounts. In some embodiments, the boronated multifunctional compounds of the present disclosure have enriched relative amounts of ¹⁰B. For example, at least 90%, at least 95%, at least 99% or more of the boron atoms in the boronated multifunctional compounds may be ¹⁰B atoms.

In various embodiments, the boronated multifunctional compounds of the present disclosure are small molecules. As used herein, a “small molecule” is one having a molecular weight of less than 2500, and in some embodiments less than 1000.

Boronated multifunctional compounds in accordance with the present disclosure include boronated polyamino compounds, which are defined herein as compounds that comprise a plurality of amino groups and one or more boron atoms.

In some embodiments, the boronated polyamino compounds comprise a polyamino moiety that is linked to a boron-containing moiety through a linking moiety, which may be selected, for example, from a bond, a linking moiety that comprises an alkyl group, a linking moiety that comprises an alkene group, a linking moiety that comprises an alkyne group, a linking moiety that comprises an ester group, a linking moiety that comprises a thioester group, a linking moiety that comprises an amide group, a linking moiety that comprises an amine group, a linking moiety that comprises an ether group, a linking moiety that comprises a carbonate group, a linking moiety that comprises a urethane group, a linking moiety that comprises a urea group, a linking moiety that comprises a linking moiety that comprises a ketone group, or a linking moiety that comprises a combination of two or more of any of the foregoing groups, among others. In some embodiments, the linking moiety comprises a hydrolysable ester group.

In some embodiments, the boronated polyamino compounds comprise a residue of a carboxylic-acid-substituted polyamino compound that is covalently linked to a residue of a boronated compound that contains one or more boron atoms.

In various embodiments, boronated polyamino compounds of the present disclosure comprise a polyamino moiety having a plurality of (e.g., two, three, four, five, six, seven, eight, nine, ten, eleven, twelve, thirteen, fourteen, fifteen, sixteen, seventeen, eighteen, nineteen, twenty or more) amino groups that is linked to a boronated moiety that contains one or more boron atoms (e.g., one, two, three, four, five, six, seven, eight, nine, ten, eleven, twelve, thirteen, fourteen, fifteen, sixteen, seventeen, eighteen, nineteen, twenty or more).

In some embodiments, the polyamino moiety comprises a plurality of (e.g., two, three, four, five, six, seven, eight, nine, ten eleven, twelve, thirteen, fourteen, fifteen, sixteen, seventeen, eighteen, nineteen, twenty or more eleven, twelve, thirteen, fourteen, fifteen, sixteen, seventeen, eighteen, nineteen, twenty or more)—(CH₂)ₓ—NH₂ groups where x is 0, 1, 2, 3, 4, 5 or 6. In some of these embodiments, the polyamino moiety may comprises a plurality of —(CH₂)ₓ—NH₂ groups disposed along a backbone of a polymer (defined herein as a moiety comprising 2, 3, 4, 5, 6, 7, 8, 9, 10 or more monomer residues). In some embodiments, the polymer backbone may be selected from a polyamide backbone, a polyalkylene backbone, or a polysaccharide backbone, among others.

Examples of carboxylic-acid-substituted polyamino compounds which can be used to form boronated polyamino compounds in accordance with the present disclosure include poly (amino acids) that comprise a plurality of primary-amine-containing side groups (e.g., 2, 3, 4, 5, 6, 7, 8, 9, 10 or more primary-amine-containing side groups), such as, for example, poly(amino acids) that comprise lysine and/or ornithine (e.g., polylysine compounds such as dilysine, trilysine, tetralysine, pentalysine, etc., polyornithine compounds such as diornithine, triornithine, tetraornithine, pentaornithine, etc., and poly(lysine-co-ornithine) compounds), as well as carboxylic-acid-terminated polyamines such as carboxylic-acid-terminated poly(allyl amine), carboxylic-acid-terminated poly(vinyl amine), or carboxylic-acid-terminated chitosan.

Examples of boronated moieties for used in the present disclosure present disclosure include including moieties that contain undecahydrododecaborate groups,

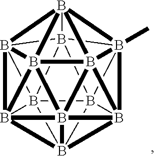

and organoborane moieties, such as dioxaborolane moieties, including moieties that contain 4,4,5,5-tetramethyl-1,3,2-dioxaborolane groups,

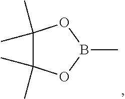

among others.

In various embodiments, boronated polyamino compounds may be formed by coupling reactions in which a carboxylic acid group of a carboxylic-acid-substituted polyamino compound, such as one of those described above, is reacted with boronated compound having a functional group that is reactive with the carboxylic acid group of the carboxylic-acid-substituted polyamino compound, for example, a thiol group, a hydroxyl group or an amino group, to form a covalent linkage, for example, a thioester-containing linkage, an ester-containing linkage or an amide-containing linkages.

Examples of boronated compounds that can be used to form boronated multifunctional compounds in accordance with the present disclosure include thiol-substituted boronated compounds, hydroxyl-substituted boronated compounds and amino-substituted boronated compounds.

Examples of thiol-substituted boronated compounds include borocaptate salts, including borocaptate sodium, also known as 1,2,3,4,5,6,7,8,9,10,11-undecahydro-12-mercapto dodecaborate(2-), sodium,

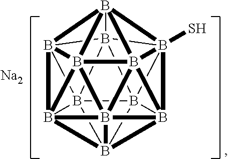

1,2,3,4,5,6,7,8,9,10,11-undecahydro-12-(mercaptomethyl) dodecaborate(2-), sodium, 1,2,3,4,5,6,7,8,9,10,11-undecahydro-12-(2-mercaptoethyl) dodecaborate(2-), sodium, or 1,2,3,4,5,6,7,8,9,10,11-undecahydro-12-(3-mercaptopropyl) dodecaborate(2-), sodium.

In some embodiments, a thioester-containing linkage may be formed by reacting the thiol group of thiol-substituted boronated compound with the carboxylic acid group of a carboxylic-acid-substituted polyamino compound in a thioester coupling reaction in the presence of a suitable coupling agent. Coupling agents include carbodiimide coupling agents such as N,N′-dicyclohexylcarbodiimide (DCC), 1-ethyl-3-(3-dimethyl‘propyl)carbodiimide (EDC), 1,3-diisopropylcarbodiimide (DIC), N-hydroxybenzotriazole (HOBt), BOP reagent, and/or another coupling agent. To prevent the amino groups of one carboxylic-acid-substituted polyamino compound from reacting with the carboxylic acid group of another carboxylic-acid-substituted polyamino compound, the amino groups of the carboxylic-acid-substituted polyamino compound may be protected using a suitable protective group. Examples of protective groups for this purpose include tert-butoxycarbonyl (Boc) groups, carboxybenzyl (CBz) or (Z) groups, trifluoroacetyl (TFA) groups, and 9-fluorenylmethoxycarbonyl (Fmoc) groups, among others. After the coupling reaction, the protective groups can be removed.

In some embodiments, a thioester-containing linkage may be formed by reacting the thiol group of thiol-substituted boronated compound with an activated-ester-substituted polyamino compound in a thioester coupling reaction. Reaction between the thiol group and the activated ester group occurs spontaneously at basic pH. After the coupling reaction, the protective groups can be removed.

An activated-ester-substituted polyamino compound may be formed from a carboxylic-acid-substituted polyamino compound by first protecting the amino groups of the carboxylic-acid-substituted polyamino compound with a suitable protective group as described above. Then, an activated ester group can be formed at the position of the carboxylic acid group. For instance, an N-hydroxy cyclic imide compound (e.g., N-hydroxysuccinimide (NHS), N-hydroxymaleimide, N-hydroxyglutarimide, N-hydroxyphthalimide, or N-hydroxy-5-norbornene-2,3-dicarboxylic acid imide, also known as N-hydroxybicyclo[2.2.1]hept-5-ene-2,3-dicarboxylic acid imide (HONB), etc.) may be reacted with the carboxylic acid group of the amine-protected carboxylic-acid-substituted polyamino compound in the presence of a suitable coupling agent, as described above, to form an activated ester group, specifically, a cyclic imide ester group (e.g., an succinimide ester group, an maleimide ester group, an glutarimide ester group, an phthalimide ester group, a diglycolimide ester group, bicyclo[2.2.1]hept-5-ene-2,3-dicarboxylic acid imide ester group, etc.).

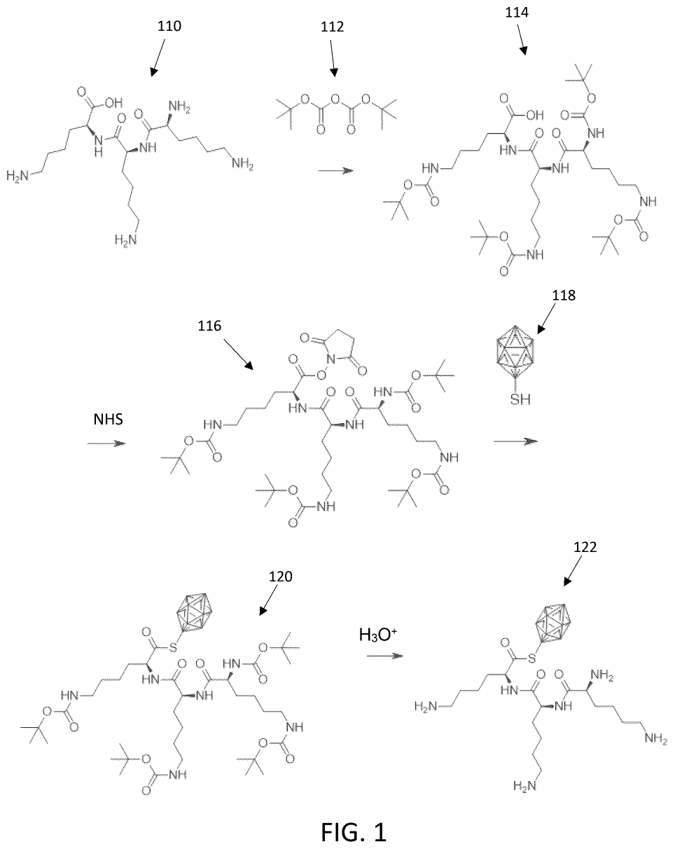

In a particular example shown in FIG. 1, amino groups of a carboxylic-acid-substituted polyamino compound, specifically, trilysine (110), are protected using di-tert-butyl dicarbonate (112), thereby forming tBoc-protected trilysine (114). This leaves the carboxyl group of the protected compound (tBoc-protected trilysine) available for further reaction. Then, an N-hydroxy cyclic imide compound, specifically, N-hydroxysuccinimide (NHS), is reacted with the carboxylic acid group of the tBoc-protected trilysine (114), in the presence of a suitable coupling agent such as DCC or DIC or EDC, to form a tBoc-protected, cyclic-imide-ester-substituted polyamino compound, specifically, tBoc-protected trilysine hydroxysuccinimide ester (116). The tBoc-protected trilysine hydroxysuccinimide ester (116) is then reacted at basic pH with borocaptate sodium (118) to form tBoc-protected trilysine undecahydrododecaborate thioester (120). In a final step, tBoc protection is removed from the tBoc-protected trilysine undecahydrododecaborate thioester (120), for example, by exposure to an acid such as HCl or trifluoroacetic acid, to provide a final boronated polyamino compound, specifically, trilysine undecahydrododecaborate thioester (120), in which an undecahydrododecaborate group is linked to a trilysine residue through a thioester group.

Examples of hydroxyl-substituted boronated compounds that can be used to form boronated multifunctional compounds in accordance with the present disclosure are hydroxyalkyl dioxaborolane compounds (e.g., C₁-C₆-hydroxyalkyl dioxaborolane compounds), including hydroxyalkyl phenyl dioxaborolane compounds (e.g., C₁-C₆-hydroxyalkyl phenyl dioxaborolane compounds). Specific examples include 4-4,4,5,5-tetramethyl-1,3,2-dioxaborolan-2-yl phenyl methanol,

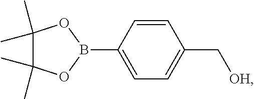 [SMILES: CC1(C)OB(c2ccc(CO)cc2)OC1(C)C]

and 4-4,4,5,5-tetramethyl-1,3,2-dioxaborolan-2-yl phenyl ethanol,

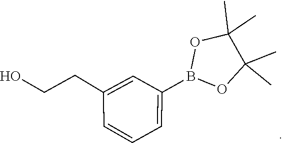 [SMILES: CC1(C)OB(c2cccc(CCO)c2)OC1(C)C]

In some embodiments, an ester-containing linkage may be formed by reacting the hydroxyl group of a hydroxyl-substituted boronated compound with the carboxylic acid group of a carboxylic-acid-substituted polyamino compound in an ester coupling reaction in the presence of a suitable coupling agent such as a carbodiimide coupling agent. Such coupling agents include carbodiimide coupling agents such as those described above. To prevent the amino groups of one carboxylic-acid-substituted polyamino compound from reacting with the carboxylic acid group of another carboxylic-acid-substituted polyamino compound, the amino groups of the carboxylic-acid-substituted polyamino compound are protected using a suitable protective group, such as those described above. After the coupling reaction, the protective groups can be removed.

Examples of amino-substituted boronated compounds that can be used to form boronated multifunctional compounds in accordance with the present disclosure are aminoalkyl dioxaborolane compounds (e.g., C₁-C₆-aminoalkyl dioxaborolane compounds), including aminoalkyl phenyl dioxaborolane compounds (e.g., C₁-C₆-aminoalkyl phenyl dioxaborolane compounds). Specific examples include (4-(4,4,5,5-tetramethyl-1,3,2-dioxaborolan-2-yl)phenyl)methanamine,

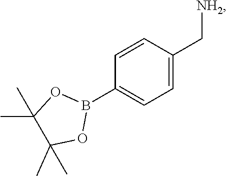 [SMILES: CC1(C)OB(c2ccc(CN)cc2)OC1(C)C]

2-(4-(4,4,5,5-tetramethyl-1,3,2-dioxaborolan-2-yl)phenyl)ethan-1-amine,

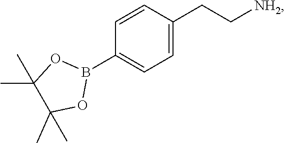 [SMILES: CC1(C)OB(c2ccc(CCN)cc2)OC1(C)C]

2-(4-(4,4,5,5-tetramethyl-1,3,2-dioxaborolan-2-yl)phenyl)ethan-2-amine, 3-(4-(4,4,5,5-tetramethyl-1,3,2-dioxaborolan-2-yl)phenyl)propan-1-amine,

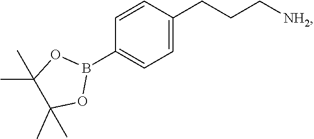 [SMILES: CC1(C)OB(c2ccc(CCCN)cc2)OC1(C)C]

3-(4-(4,4,5,5-tetramethyl-1,3,2-dioxaborolan-2-yl)phenyl)propan-2-amine, 2-((4-(4,4,5,5-tetramethyl-1,3,2-dioxaborolan-2-yl)benzyl)oxy)ethan-1-amine,

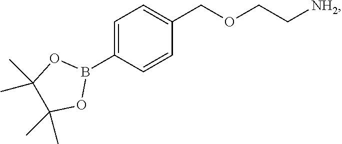 [SMILES: CC1(C)OB(c2ccc(COCCN)cc2)OC1(C)C]

and 4-(4,4,5,5-Tetramethyl-1,3,2-dioxaborolan-2-yl)benzyl (2-aminoethyl)carbamate,

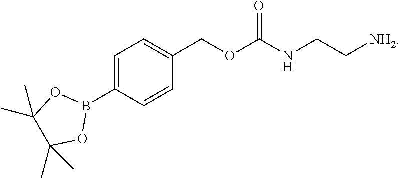 [SMILES: CC1(C)OB(c2ccc(COC(=O)NCCN)cc2)OC1(C)C]

In some embodiments, an amide-containing linkage may be formed by reacting the amino group of an amino-substituted boronated compound with the carboxylic acid group of a carboxylic-acid-substituted polyamino compound in an amide coupling reaction in the presence of a suitable coupling agent such as a carbodiimide coupling agent. Such coupling agents include carbodiimide coupling agents such as those described above. To prevent the amino groups of prevent the amino groups of one carboxylic-acid-substituted polyamino compound from reacting with the carboxylic acid group of another carboxylic-acid-substituted polyamino compound, the amino groups of the carboxylic-acid-substituted polyamino compound are protected using a suitable protective group, such as those described above. After the coupling reaction, the protective groups can be removed.

As previously noted, the above-described boronated multifunctional compounds are useful as crosslinkers for reactive polymers that have a plurality of electrophilic groups, which are reactive with the nucleophilic groups of the boronated multifunctional compound.

As also previously noted, electrophilic groups may be selected, for example, from cyclic imide ester groups, such as succinimide ester groups,

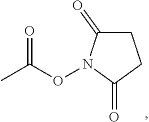

maleimide ester groups, glutarimide ester groups, diglycolimide ester groups, phthalimide ester groups, and bicyclo[2.2.1]hept-5-ene-2,3-dicarboxylic acid imide ester groups,

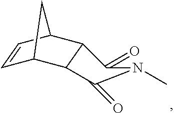

imidazole ester groups, imidazole carboxylate groups and benzotriazole ester groups, among other possibilities.

Reactive polymers in accordance with the present disclosure include reactive multi-arm polymers. In some embodiments, reactive multi-arm polymers in accordance with the present disclosure comprise a plurality of polymer arms linked to a core region, where the polymer arms comprise a hydrophilic polymer segment. One end of the hydrophilic polymer segment is covalently attached to the core region through a suitable linkage, and an electrophilic group is covalently attached to an opposite end of the hydrophilic polymer segment through a suitable linkage.

Reactive multi-arm polymers in accordance with the present disclosure include polymers having from 2 to 100 arms, for example ranging anywhere from 2 to 3 to 4 to 5 to 6 to 7 to 8 to 9 to 10 to 11 to 12 to 15 to 20 to 25 to 50 to 75 to 100 arms (in other words, having a number of arms ranging between any two of the preceding values).

The electrophilic groups may be linked to the hydrophilic polymer segment and the hydrophilic polymer segment may be linked to the core through any suitable linking moiety, which may be selected, for example, from a bond, a linking moiety that comprises a bond, an alkyl group, a linking moiety that comprises an alkene group, a linking moiety that comprises an alkyne group, a linking moiety that comprises an ether group, a linking moiety that comprises an ester group, a linking moiety that comprises an amide group, a linking moiety that comprises an amine group, a linking moiety that comprises a carbonate group, a linking moiety that comprises a urethane group, a linking moiety that comprises a urea group, a linking moiety that comprises a ketone group, or a linking moiety that comprises a combination of two or more of any of the foregoing groups, among others. In various embodiments, the linking moiety comprises a hydrolysable ester group.

Hydrophilic polymer segments can be selected from any of a variety of synthetic, natural, or hybrid synthetic-natural hydrophilic polymer segments. Examples of hydrophilic polymer segments include those that are formed from one or more hydrophilic monomers selected from the following: C₁-C₆-alkylene oxides (e.g., ethylene oxide, propylene oxide, tetramethylene oxide, etc.), polar aprotic vinyl monomers (e.g. N-vinyl pyrrolidone, acrylamide, N-methyl acrylamide, dimethyl acrylamide, N-vinyl imidazole, 4-vinylimidazole, sodium 4-vinylbenzenesulfonate, etc.), dioxanone, ester monomers (e.g. glycolide, lactide, β-propiolactone, β-butyrolactone, γ-butyrolactone, γ-valerolactone, δ-valerolactone, ε-caprolactone, etc.), oxazoline monomers (e.g., oxazoline and 2-alkyl-2-oxazolines, for instance, 2-(C₁-C₆ alkyl)-2-oxazolines, including various isomers, such as 2-methyl-2-oxazoline, 2-ethyl-2-oxazoline, 2-n-propyl-2-oxazoline, 2-isopropyl-2-oxazoline, 2-n-butyl-2-oxazoline, 2-isobutyl-2-oxazoline, 2-hexyl-2-oxazoline, etc.), 2-phenyl-2-oxazoline, N-isopropylacrylamide, amino acids and sugars.

Hydrophilic polymer segments may be selected, for example, from the following polymer segments: polyether segments including poly (C₁-C₆-alkylene oxide) segments such as poly (ethylene oxide) (PEO) (also referred to as polyethylene glycol or PEG) segments, poly(propylene oxide) segments, poly(ethylene oxide-co-propylene oxide) segments, polymer segments formed from one or more polar aprotic vinyl monomers, including poly(N-vinyl pyrrolidone) segments, poly(acrylamide) segments, poly(N-methyl acrylamide) segments, poly(dimethyl acrylamide) segments, poly(N-vinylimidazole) segments, poly(4-vinylimidazole) segments, and poly(sodium 4-vinylbenzenesulfonate) segments, polydioxanone segments, polyester segments including polyglycolide segments, polylactide segments, poly(lactide-co-glycolide) segments, poly(β-propiolactone) segments, poly(β-butyrolactone) segments, poly(γ-butyrolactone) segments, poly(γ-valerolactone) segments, poly(δ-valerolactone) segments, and poly(ε-caprolactone) segments, polyoxazoline segments including poly(2-C₁-C₆-alkyl-2-oxazoline segments) such as poly(2-methyl-2-oxazoline) segments, poly(2-ethyl-2-oxazoline) segments, poly(2-propyl-2-oxazoline) segments, poly(2-isopropyl-2-oxazoline) segments, and poly(2-n-butyl-2-oxazoline) segments, poly(2-phenyl-2-oxazoline) segments, poly(N-isopropylacrylamide) segments, polypeptide segments, and polysaccharide segments. Polysaccharide segments include those that contain one or more uronic acid species, such as galacturonic acid, glucuronic acid and/or iduronic acid, with particular examples of polysaccharide segments including alginic acid, hyaluronic acid, pectin, agaropectin, carrageenan, gellan gum, gum arabic, guar gum, xanthan gum, and carboxymethyl cellulose moieties.

Polymer segments for use in the multi-arm polymers of the present disclosure typically contain from 10 monomer units or less to 1000 monomer units or more, for example, ranging anywhere from 5 to 10 to 20 to 50 to 100 to 200 to 500 to 1000 to 2000 monomer units.

In certain embodiments, the core region comprises a residue of a polyhydroxy compound comprising two or more hydroxyl groups, also referred to herein as a polyol. Polyols for use in the present disclosure may have two, three, four, five, six, seven, eight, nine, ten, eleven, twelve, thirteen, fourteen, fifteen, sixteen, seventeen, eighteen, nineteen, twenty, twenty-five, thirty or more hydroxyl groups. In various embodiments, polyols for use in the present disclosure may have further hydrophilic groups in addition to hydroxyl groups, including ether groups, amine groups, ester groups, amide groups.

Polyols for use in the present disclosure include non-iodinated and iodinated polyols.

Polyols may be selected, for example, from non-iodinated and iodinated sugars (monosaccharides, disaccharides, trisaccharides, etc.), sugar alcohols, calixarenes, cyclodextrins, polyhydroxylated polymers, catechins, flavanols, anthocyanins, stilbenes, and polyphenols, among others.

Non-iodinated polyols may be selected, for example, from straight-chained, branched and cyclic aliphatic polyols including straight-chained, branched and cyclic polyhydroxyalkanes, straight-chained, branched and cyclic polyhydroxy ethers, including polyhydroxy polyethers, straight-chained, branched and cyclic polyhydroxyalkyl ethers, including polyhydroxyalkyl polyethers, straight-chained, branched and cyclic sugars and sugar alcohols. Specific examples include methane triol, glycerol, trimethylolpropane, benzenetriol, mannitol, sorbitol, inositol, xylitol, quebrachitol, threitol, arabitol, erythritol, pentaerythritol, dipentaerythritol, tripentaerythritol, adonitol, hexaglycerol, dulcitol, fucose, ribose, arabinose, xylose, lyxose, rhamnose, galactose, glucose, fructose, sorbose, mannose, pyranose, altrose, talose, tagatose, pyranosides, sucrose, lactose, and maltose, polymers (defined herein as two or more units) of straight-chained, branched and cyclic sugars and sugar alcohols, including oligomers (defined herein as ranging from two to ten units, including dimers, trimers, tetramers, pentamers, hexamers, heptamers, octamers, enneamers and decamers) of straight-chained, branched and cyclic sugars and sugar alcohols, including the preceding sugars and sugar alcohols, starches, amylose, dextrins, cyclodextrins, catechins, flavanols, anthocyanins, stilbenes, polyphenols, as well as polyhydroxy crown ethers, and polyhydroxyalkyl crown ethers. Illustrative polyols also include aromatic polyols including 1,1,1-tris(4′-hydroxyphenyl) alkanes, such as 1,1,1-tris(4-hydroxyphenyl)ethane, and 2,6-bis(hydroxyalkyl)cresols, among others.

Illustrative non-iodinated polyols also include polyhydroxylated polymers such as poly(vinyl alcohol), poly(allyl alcohol), poly(hydroxyethyl acrylate), or poly(hydroxyethyl methacrylate), among others. Such polyhydroxylated polymers may range, for example, from 2 to 100 monomer units in length.

Iodinated polyols may be desirable where radiopacity is desired. Iodinated polyols include iodinated aromatic polyols, examples of which are compounds that comprise two or more hydroxyl groups (e.g., two, three, four, five, six, seven, eight, nine, ten, eleven, twelve or more hydroxyl groups), and one or more iodinated aromatic groups (e.g., one, two, three, four, five, six or more iodine atoms). Examples of iodinated aromatic groups include iodine-substituted monocyclic aromatic groups and iodine-substituted multicyclic aromatic groups which may contain, for example, six, ten, fourteen, eighteen or more carbon atoms, such as iodine-substituted phenyl groups, iodine-substituted naphthyl groups, iodine-substituted anthracenyl groups, iodine-substituted phenanthrenyl groups and iodine-substituted tetracenyl groups, among others. The aromatic groups may be substituted with one, two, three, four, five, six or more iodine atoms. In various embodiments, the aromatic groups are further substituted with two or more hydroxyl groups, which may be directly substituted to the aromatic groups or may be provided in the form of hydroxyalkyl groups (e.g., C₁-C₄-hydroxyalkyl groups containing one, two, three or four carbon atoms and containing one, two, three or four or more hydroxyl groups). The hydroxyalkyl groups may be linked to the aromatic group directly or through any suitable linking moiety, which may be selected, for example, from amide groups, ether groups, alkyl groups, amine groups, and combinations thereof, among others.

Specific examples of iodinated polyols for use in the present disclosure include commercially available 1,3,5-triiodo-2,4,6-trishydroxymethylbenzene, iodixanol, iotrolan, iohexol, ioversol, iopamidol, iopamidol, iomeprol, iobitridol, iohexol impurity J, metrizamide, ioxilan, iopentol, and iopromide, among others.

Further illustrative non-iodinated polyols include silsesquioxanes, which are compounds that have a cage-like silicon-oxygen core that is made up of Si—O—Si linkages and tetrahedral Si vertices. exterior organic groups may be covalently attached to the cage-like silicon-oxygen core. In the present disclosure, the organic groups comprise one or more hydroxyl groups. Silsesquioxanes for use in the present disclosure include silsesquioxanes with 6 Si vertices, silsesquioxanes with 8 Si vertices, silsesquioxanes with 10 Si vertices, and silsesquioxanes with 12 Si vertices. The silicon-oxygen cores are sometimes referred to as T6, T8, T10, and T12 cage-like silicon-oxygen cores, respectively (where T=the number of tetrahedral Si vertices). In all cases each Si atom is bonded to three O atoms, which in turn connect to other Si atoms. Silsesquioxanes include compounds of the chemical formula [RSiO_{3/2}]ₙ, where n is an integer of at least 6, commonly 6, 8, 10 or 12 (thereby having T₆, T₈, T₁₀ or T₁₂ cage-like silicon-oxygen core, respectively), and where R is an organic group that comprises one or more hydroxyl groups. The T₈ cage-like silicon-oxygen cores are widely studied and have the formula [RSiO_{3/2}]₈, or equivalently R₈Si₈O₁₂. Such a structure is shown here:

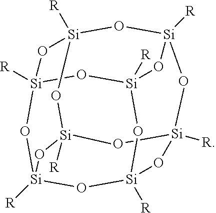

In the present disclosure, the R groups are organic groups that comprise one or more hydroxyl groups. Examples of organic groups include hydroxyalkyl groups, for example, C₁-C₄-hydroxyalkyl groups containing one, two, three or four carbon atoms and containing one, two, three or four or more hydroxyl groups, among others.

Reactive multi-arm polymers in accordance with the present disclosure can be formed from hydroxy-terminated multi-arm polymers having arms that comprise one or more hydroxyl end groups. In some embodiments of the present disclosure, a polyol such as one of those described above, among others, may be used as a multi-functional initiator for polymer chain growth. For example, the polyol may be used as an initiator for ring-opening polymerization of ethylene oxide to form polyethylene oxide (PEO) segments (also referred to a polyethylene glycol, or PEG, segments) at each of the hydroxyl groups of the polyol. The resulting hydroxyl-terminated PEG segments possess tunable hydrophilicity depending on the desired water-solubility of the resulting multi-arm polymer, for example, with increasing PEG segment length leading to increasing hydrophilicity. Hydroxyl-terminated multi-arm polymers are also available commercially. For example, hydroxyl-terminated four-arm PEG, hydroxyl-terminated six-arm PEG, and hydroxyl-terminated eight-arm PEG are available from JenKem Technology USA, Plano, TX, U.S.A.

In some embodiments, a hydroxy-terminated multi-arm hydrophilic polymer may be reacted with a cyclic anhydride to form carboxylic-acid-terminated polymer in which carboxylic acid end groups are linked to hydrophilic polymer segments through hydrolysable ester groups. For example, terminal hydroxyl groups of the hydrophilic polymer segments may be reacted with a cyclic anhydride (e.g., glutaric anhydride, succinic anhydride, malonic anhydride, adipic anhydride, diglycolic anhydride, etc.) to form a carboxylic-acid-terminated segment such as a glutaric-acid-terminated segment, a succinic-acid-terminated segment, a malonic-acid-terminated segment, an adipic-acid-terminated segment, a diglycolic-acid-terminated segment, and so forth.

The preceding cyclic anhydrides, among others, may be reacted with a hydroxy-terminated multi-arm hydrophilic polymer under basic conditions to form a carboxylic-acid-terminated multi-arm hydrophilic polymer comprising a carboxylic acid end group that is linked to a hydrophilic polymer segment through a hydrolysable ester group. Carboxylic-acid-terminated multi-arm polymers are also available commercially. For example, carboxylic-acid-terminated four-arm PEG and carboxylic-acid-terminated eight-arm PEG (without hydrolysable ester groups) are available from JenKem Technology USA.

An electrophilic moiety, such as a cyclic-imide-containing moiety, may be linked to the carboxylic-acid-terminated multi-arm hydrophilic polymer. For instance, an N-hydroxy cyclic imide compound (e.g., N-hydroxysuccinimide, N-hydroxymaleimide, N-hydroxyglutarimide, N-hydroxyphthalimide, or N-hydroxy-5-norbornene-2,3-dicarboxylic acid imide, also known as N-hydroxybicyclo[2.2.1]hept-5-ene-2,3-dicarboxylic acid imide (HONB), etc.) may be reacted with the carboxylic-acid-terminated multi-arm hydrophilic polymer in the presence of a suitable coupling agent (e.g., a carbodiimide coupling agent) to form an activated ester group, in particular, a cyclic imide ester group (e.g., an succinimide ester group, an maleimide ester group, an glutarimide ester group, an phthalimide ester group, a diglycolimide ester group, bicyclo[2.2.1]hept-5-ene-2,3-dicarboxylic acid imide ester group, etc.) that is linked to a hydrophilic polymer segment through a hydrolysable ester group. In this way, a number of reactive diester groups can be formed. For example, in the particular case of N-hydroxysuccinimide as an N-hydroxy cyclic imide compound, exemplary reactive diester groups include succinimidyl malonate groups, succinimidyl glutarate groups, succinimidyl succinate groups, succinimidyl adipate groups, and succinimidyl diglycolate groups, among others. In the particular case of HONB as an N-hydroxy cyclic imide compound, exemplary reactive diester groups include bicyclo[2.2.1]hept-5-ene-2,3-dicarboxylic acid imidyl malonate groups, bicyclo[2.2.1]hept-5-ene-2,3-dicarboxylic acid imidyl glutarate groups, bicyclo[2.2.1]hept-5-ene-2,3-dicarboxylic acid imidyl succinate groups, bicyclo[2.2.1]hept-5-ene-2,3-dicarboxylic acid imidyl adipate groups, and bicyclo[2.2.1]hept-5-ene-2,3-dicarboxylic acid imidyl diglycolate groups, among others. In the particular case of N-hydroxymaleimide as an N-hydroxy cyclic imide compound, exemplary reactive diester groups include maleimidyl malonate groups, maleimidyl glutarate groups, maleimidyl succinate groups, maleimidyl adipate groups, and maleimidyl diglycolate groups, among others. In the particular case of N-hydroxyglutarimide as an N-hydroxy cyclic imide compound, exemplary reactive diester groups include glutarimidyl malonate groups, glutarimidyl glutarate groups, glutarimidyl succinate groups, glutarimidyl adipate groups, glutarimidyl diglycolate groups, among others. In the particular case of N-hydroxyphthalimide as an N-hydroxy cyclic imide compound, exemplary reactive diester groups include phthalimidyl malonate groups, phthalimidyl glutarate groups, phthalimidyl succinate groups, phthalimidyl adipate groups, and phthalimidyl diglycolate groups, among others. Some multi-arm polymers having reactive diester end groups are also available commercially. For example, succinimidyl-glutarate-terminated four-arm PEG and succinimidyl-glutarate-terminated eight-arm PEG are available from JenKem Technology USA.

In other aspects, the present disclosure provides crosslinked reaction products of (a) a boronated multifunctional compound comprising a plurality of nucleophilic groups, such as those described herein and (b) a reactive polymer that comprises a plurality of electrophilic groups, such as those described herein.

Such crosslinked products may be formed in vivo (e.g., using a delivery device like that described below), or such crosslinked products may be formed ex vivo and subsequently administered to a subject.

In various embodiments, the crosslinked reaction products are hydrogels. As used herein, a “hydrogel,” which may also be referred to herein as a “crosslinked hydrogel,” is a crosslinked polymer that contains water or can absorb water but does not dissolve when placed in water.

In some embodiments, the crosslinked products are visible under fluoroscopy. The crosslinked hydrogels may have a radiopacity that is greater than 100 Hounsfield units (HU), beneficially anywhere ranging from 100 HU to 250 HU to 500 HU to 750 HU to 1000 HU to 2000 HU or more (in other words, ranging between any two of the preceding numerical values), for example, when measured on bench-top micro CT systems such as XtremeCT from Scanco Medical (Wangen-Brüttisellen, Switzerland) or similar.

In various embodiments, the crosslinked reaction products break down in vivo over a period ranging from 8 weeks to 26 weeks, more typically ranging from 8 weeks to 12 weeks.

In some embodiments, crosslinked reaction products are formed from (a) a boronated polyamino compound, such as those described herein, and (b) a reactive polymer that comprises a plurality of cyclic imide ester groups, such as those described herein, wherein the cyclic imide ester groups of the reactive polymer and the amino groups of the boronated polyamino compound react with one another via an amide coupling reaction to form a crosslinked product.

In some aspects of the present disclosure, systems are provided that are configured to deliver (a) a boronated multifunctional compound comprising a plurality of nucleophilic groups, such as those described herein, and (b) a reactive polymer that comprises a plurality of electrophilic groups, such as those described herein. The boronated multifunctional compound and the reactive polymer are combined under conditions such that the nucleophilic groups of the boronated multifunctional compound and the electrophilic end groups of the reactive polymer crosslink with one another. Such systems can be used to form crosslinked hydrogels, either in vivo or ex vivo.

For example, in some embodiments systems are provided that are configured to deliver (a) a boronated polyamino compound as described herein and (b) a reactive polymer comprising a plurality of cyclic imide ester groups as described herein. The boronated polyamino compound and the reactive polymer are combined under conditions such that the amino groups of the boronated polyamino compound and the cyclic imide ester groups of the reactive polymer crosslink with one another. In certain embodiments, those conditions comprise an environment having a basic pH, for example, a pH ranging from about 8.5 to about 12. Such systems can be used to form crosslinked hydrogels, either in vivo or ex vivo.

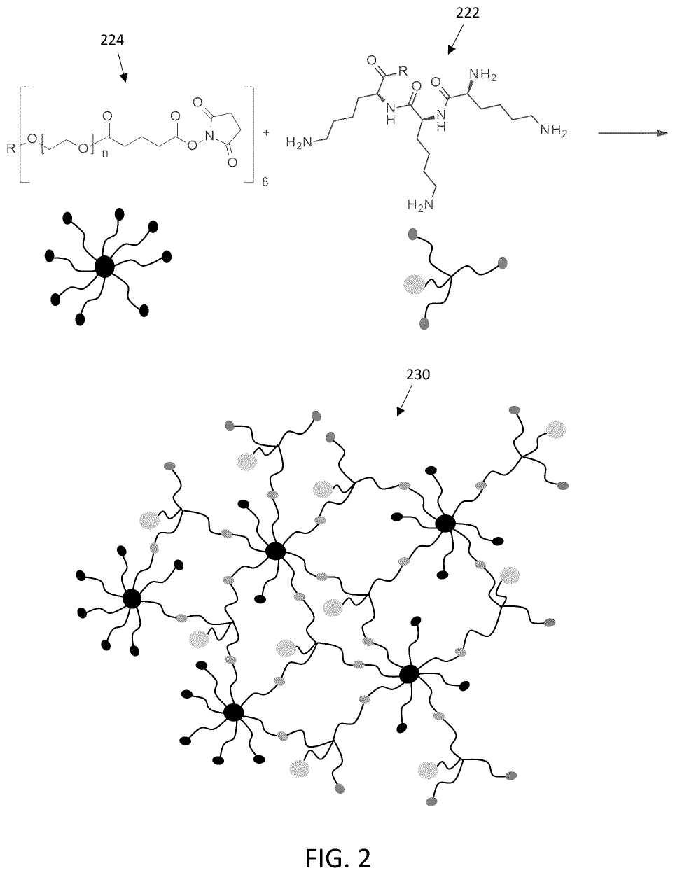

A particular example of a crosslinking reaction is illustrated in FIG. 2, which shows a covalent crosslinking reaction between (a) amino groups of a boronated polyamino compound (222), specifically, a comprising a trilysine residue and residue of a boronated polyamino compound, designated by an R group and (b) cyclic amide ester groups of a reactive polymer (224), specifically, succinimide ester groups a succinimidyl-glutarate-terminated eight-arm PEG molecule, where R corresponds to a core, for example, a polyol residue core such as a hexaglycerol residue core or a tripentaerythritol residue core (although only one polymer arm of the multi-arm polymer is shown attached to the core R, it is to be understood that additional polymer arms are present), and where n is an integer and may have a value ranging from 5 to 1000 or more. The crosslinking reaction between the amino groups of the boronated polyamino compound (222) and the cyclic amide ester groups of the reactive polymer (224) results in the formation of amide linking groups which act as crosslinks for the resulting crosslinked reaction product (230). The crosslinking reaction shown is inhibited at acidic pH, but occurs spontaneously at basic pH.

In some aspects of the present disclosure, a system is provided that comprises (a) a first composition that comprises a boronated multifunctional compound as described herein and (b) a second composition that comprises a reactive polymer as described herein.

The first composition may be a first fluid composition comprising the boronated multifunctional compound or a first dry composition that comprises the boronated multifunctional compound, to which a suitable fluid such as water for injection, saline, etc. can be added to form a first fluid composition. In addition to the boronated multifunctional compound, the first composition may further comprise additional agents, including therapeutic agents, imaging agents, colorants, tonicity adjusting agents, suspension agents, wetting agents, and pH adjusting agents as described below.

The second composition may be a second fluid composition comprising the reactive polymer or a second dry composition that comprises the reactive polymer, to which a suitable fluid such as water for injection, saline, etc. can be added to form a second fluid composition. In addition to the reactive polymer, the second composition may further comprise additional agents, including therapeutic agents, imaging agents, colorants, tonicity adjusting agents, suspension agents, wetting agents, and pH adjusting agents as described below.

In some embodiments, the boronated multifunctional compound is initially combined with the reactive polymer under conditions where crosslinking between the electrophilic moieties of the reactive polymer and the amino groups of the boronated multifunctional compound is suppressed (e.g., an acidic pH, in some embodiments). Then, when crosslinking is desired, the conditions are changed such that crosslinking is increased (e.g., a change from an acidic pH to a basic pH, in some embodiments), leading to crosslinking between same, thereby forming the crosslinked product.

In some embodiments, the system comprises (a) a first composition that comprises a boronated multifunctional compound as described hereinabove, (b) a second composition that comprises a reactive polymer as described hereinabove, and (c) a third composition, specifically, an accelerant composition, that contains an accelerant that is configured to accelerate a crosslinking reaction between the boronated multifunctional compound and the reactive polymer.

The first composition may be (a) a first fluid composition comprising the boronated multifunctional compound that is buffered to an acidic pH or (b) a first dry composition that comprises the boronated multifunctional compound and acidic buffering composition, to which a suitable fluid such as water for injection, saline, etc. can be added to form a first fluid composition comprising the boronated multifunctional compound that is buffered to an acidic pH. In some embodiments, for example, the acidic buffering composition may comprise monobasic sodium phosphate, among other possibilities. The first fluid composition comprising the boronated multifunctional compound may have a pH ranging, for example, from about 3 to about 6.5. In addition to the boronated multifunctional compound, the first composition may further comprise additional agents, including therapeutic agents, imaging agents, colorants, tonicity adjusting agents, suspension agents, wetting agents, and pH adjusting agents as described below.

The second composition may be a second fluid composition comprising the reactive polymer or a second dry composition that comprises the reactive polymer from which a fluid composition is formed, for example, by the addition of a suitable fluid such as water for injection, saline, or the first fluid composition comprising the boronated multifunctional compound that is buffered to an acidic pH. In addition to the reactive polymer, the second composition may further comprise additional agents, including therapeutic agents, imaging agents, colorants, tonicity adjusting agents, suspension agents, wetting agents, and pH adjusting agents as described below.

In a particular embodiment, the first composition is a first fluid composition comprising the boronated multifunctional compound that is buffered to an acidic pH and the second composition comprises a dry composition that comprises the reactive polymer. The first composition may then be mixed with the second composition to provide a prepared fluid composition that is buffered to an acidic pH and comprises the boronated multifunctional compound and the reactive polymer. In a particular example, a syringe may be provided that contains the first fluid composition comprising the boronated multifunctional compound that is buffered to an acidic pH, and a vial may be provided that comprises the dry composition (e.g., a powder) that comprises the reactive polymer. The syringe may then be used to inject the first fluid composition into the vial containing the reactive polymer to form a prepared fluid composition that contains the boronated multifunctional compound and the reactive polymer, which can be withdrawn back into the syringe for administration.

In some embodiments, the system comprises (a) a first composition that comprises a boronated multifunctional compound as described hereinabove and a reactive polymer as described hereinabove and, optionally, one or more additional agents as described below, (b) a second composition, in the form of a fluid that is buffered to an acidic pH (the acidic pH hindering a crosslinking reaction between the boronated multifunctional compound and the reactive polymer), or in the form of a dry composition that comprises an acidic buffering composition to which a suitable fluid such as water for injection, saline, etc. can be added to form a fluid that is buffered to an acidic pH, and, optionally, one or more additional agents as described below and (c) a third composition, specifically, an accelerant composition, that contains an accelerant that is configured to accelerate a crosslinking reaction between the boronated multifunctional compound and the reactive polymer and, optionally, one or more additional agents as described below.

In a particular embodiment, the first composition that comprises the boronated multifunctional compound and the reactive polymer is a dry composition (e.g., a powder) and the second composition is in the form of the fluid that is buffered to an acidic pH. The dry first composition and the fluid second composition may then be mixed to provide a prepared fluid composition that is buffered to an acidic pH and comprises the boronated multifunctional compound and the reactive polymer (and optional additional agents). In a particular example, a vial may be provided that comprises the dry first composition and a syringe may be provided that contains the fluid second composition that is buffered to an acidic pH. The syringe may then be used to inject the fluid second composition into the vial containing the boronated multifunctional compound and the reactive polymer to form a prepared fluid composition that is buffered to an acidic pH and contains the boronated multifunctional compound and the reactive polymer, which can be withdrawn back into the syringe for administration.

The third composition may be a fluid accelerant composition that is buffered to a basic pH or a dry composition that comprises a basic buffering composition to which a suitable fluid such as water for injection, saline, etc. can be added to form a fluid accelerant composition that is buffered to a basic pH. For example, the basic buffering composition may comprise sodium borate and dibasic sodium phosphate, among other possibilities. The fluid accelerant composition may have, for example, a pH ranging from about 9 to about 11. In addition to the above, the fluid accelerant composition may further comprise additional agents, including those described below.

A prepared fluid composition that is buffered to an acidic pH and comprises the boronated multifunctional compound and the reactive polymer as described above (as well as additional agents in some cases), and a fluid accelerant composition that is buffered to basic pH as described above (which may include additional agents in some cases), may be combined to form crosslinked hydrogels, either in vivo or ex vivo.

In some particular embodiments of the present disclosure, a kit is provided that include a first reservoir (e.g., a vial or syringe barrel) containing a first composition comprising a boronated multifunctional compound as described herein and an acidic buffer, a second reservoir (e.g., a vial or syringe barrel) containing a second composition comprising containing a reactive polymer as described herein, a third reservoir (e.g., a vial or syringe barrel) containing a third composition comprising a buffered accelerant as described herein, additional apparatus, as required, for combining the first and second compositions to provide a prepared fluid composition that is buffered to an acidic pH and comprises the boronated multifunctional compound and the reactive polymer, and additional apparatus for combining and delivering the prepared fluid composition and buffered accelerant to a patient, for example, a double-barrel syringe device that combines the prepared fluid composition and the buffered accelerant and one or more of a tube, a sprayer (which may be connected to the double-barrel syringe device, for example, directly or through the tube) and a needle (which may be connected to the double-barrel syringe device, for example, directly or through the tube). In some cases, the kit may further include an injectable liquid such as water for injection, normal saline or phosphate buffered saline.

In more particular embodiments, the kit may comprise a vial containing a reactive polymer as described herein in dry (e.g., powdered) form, a first syringe containing a fluid composition comprising a boronated multifunctional compound as described herein that is buffered to an acidic pH, a second syringe containing a buffered accelerant solution as described herein, a needle and/or tube, a Y-connector, a syringe holder, a plunger cap and a vial adapter. Such components may be placed in sterile packaging, for example, in one or more packaged sterile trays.

In some particular embodiments of the present disclosure, a kit is provided that include a first reservoir (e.g., a vial or syringe barrel) containing a first composition comprising a boronated multifunctional compound as described herein and a reactive polymer as described herein, a second reservoir (e.g., a vial or syringe barrel) containing a second composition comprising containing an acidic buffer as described herein, a third reservoir (e.g., a vial or syringe barrel) containing a third composition comprising a buffered accelerant as described herein, additional apparatus, as required, for combining the first and second compositions to provide a prepared fluid composition that is buffered to an acidic pH and comprises the boronated multifunctional compound and the reactive polymer, and additional apparatus for combining and delivering the prepared fluid composition and buffered accelerant to a patient, for example, a double-barrel syringe device that combines the prepared fluid composition and the buffered accelerant and one or more of a tube, a sprayer (which may be connected to the double-barrel syringe device, for example, directly or through the tube) and a needle (which may be connected to the double-barrel syringe device, for example, directly or through the tube). In some cases, the kit may further include an injectable liquid such as water for injection, normal saline or phosphate buffered saline.

In more particular embodiments, the kit may comprise a vial containing a reactive polymer and reactive polymer as described herein in dry (e.g., powdered) form, a first syringe containing a fluid composition comprising a solution that is buffered to an acidic pH, a second syringe containing a buffered accelerant solution as described herein, a needle and/or tube, a Y-connector, a syringe holder, a plunger cap and a vial adapter. Such components may be placed in sterile packaging, for example, in one or more packaged sterile trays.

The compositions described herein may be sterilized using any suitable method. For example, the compositions may be autoclaved while inside a reservoir, such as a syringe barrel, vial, or ampule by heating the mixture at or to a temperature of about 121° C. Alternatively or additionally, the compositions may be sterilized via sterile filtration and/or by supercritical CO₂, gamma, x-ray or electron beam irradiation.

Additional agents for use in the compositions described herein include therapeutic agents, imaging agents, colorants, tonicity adjusting agents, suspension agents, wetting agents, and pH adjusting agents.

Examples of therapeutic agents include antithrombotic agents, anticoagulant agents, antiplatelet agents, thrombolytic agents, antiproliferative agents, anti-inflammatory agents, hyperplasia inhibiting agents, anti-restenosis agent, smooth muscle cell inhibitors, antibiotics, antimicrobials, analgesics, anesthetics, growth factors, growth factor inhibitors, cell adhesion inhibitors, cell adhesion promoters, anti-angiogenic agents, cytotoxic agents, chemotherapeutic agents, checkpoint inhibitors, immune modulatory cytokines, T-cell agonists, STING (stimulator of interferon genes) agonists, antimetabolites, alkylating agents, microtubule inhibitors, hormones, hormone antagonists, monoclonal antibodies, antimitotics, immunosuppressive agents, tyrosine and serine/threonine kinases, proteasome inhibitors, matrix metalloproteinase inhibitors, Bcl-2 inhibitors, DNA alkylating agents, spindle poisons, poly (DP-ribose)polymerase (PARP) inhibitors, and combinations thereof.

Examples of imaging agents include (a) fluorescent dyes such as fluorescein, indocyanine green, or fluorescent proteins (e.g. green, blue, cyan fluorescent proteins), (b) contrast agents for use in conjunction with magnetic resonance imaging (MRI), including contrast agents that contain elements that form paramagnetic ions, such as Gd (III), Mn (II), Fe (III) and compounds (including chelates) containing the same, such as gadolinium ion chelated with diethylenetriaminepentaacetic acid, (c) contrast agents for use in conjunction with ultrasound imaging, including organic and inorganic echogenic particles (i.e., particles that result in an increase in the reflected ultrasonic energy) or organic and inorganic echolucent particles (i.e., particles that result in a decrease in the reflected ultrasonic energy), (d) contrast agents for use in connection with near-infrared (NIR) imaging, which can be selected to impart near-infrared fluorescence to the hydrogels of the present disclosure, allowing for deep tissue imaging and device marking, for instance, NIR-sensitive nanoparticles such as gold nanoshells, carbon nanotubes (e.g., nanotubes derivatized with hydroxy or carboxylic acid groups, for instance, partially oxidized carbon nanotubes), dye-containing nanoparticles, such as dye-doped nanofibers and dye-encapsulating nanoparticles, and semiconductor quantum dots, among others, and NIR-sensitive dyes such as cyanine dyes, squaraines, phthalocyanines, porphyrin derivatives and boron dipyrromethane (BODIPY) analogs, among others, (e) imageable radioisotopes including 99mTc, 201Th, 51Cr, 67Ga, 68Ga, 111In, 64Cu, 89Zr, 59Fe, 42K, 82Rb, 24Na, 45Ti, 44Sc, 51Cr and 177Lu, among others, and (f) radiocontrast agents such as metallic particles, for example, particles of tantalum, tungsten, rhenium, niobium, molybdenum, and their alloys, which metallic particles may be spherical or non-spherical. Additional examples of radiocontrast agents include non-ionic radiocontrast agents, such as iohexol, iodixanol, ioversol, iopamidol, ioxilan, or iopromide, ionic radiocontrast agents such as diatrizoate, iothalamate, metrizoate, or ioxaglate, and iodinated oils, including ethiodized poppyseed oil (available as Lipiodol®).

Examples of colorants include brilliant blue (e.g., Brilliant Blue FCF, also known as FD &C Blue 1), indigo carmine (also known as FD&C Blue 2), indigo carmine lake, FD&C Blue 1 lake, and methylene blue (also known as methylthioninium chloride), among others.

Examples of additional agents further include tonicity adjusting agents such as sugars (e.g., dextrose, lactose, etc.), polyhydric alcohols (e.g., glycerol, propylene glycol, mannitol, sorbitol, etc.) and inorganic salts (e.g., potassium chloride, sodium chloride, etc.), among others, suspension agents including various surfactants, wetting agents, and polymers (e.g., albumen, PEO, polyvinyl alcohol, block polymers, etc.), among others, and pH adjusting agents including various buffer solutes.

A prepared fluid composition that is buffered to an acidic pH and comprises the boronated multifunctional compound and the reactive polymer as described above, and a fluid accelerant composition that is buffered to basic pH as described above, may be combined to form crosslinked hydrogels, either in vivo or ex vivo.

In various embodiments, a system is provided that includes one or more delivery devices for delivering first and second compositions to a subject.

In some embodiments, the system may include a delivery device that comprises a first reservoir that contains a first composition that comprises a boronated multifunctional compound as described above and a second reservoir that contains a second composition that comprises a reactive polymer that comprises a plurality of electrophilic moieties that are reactive with the amino moieties of the boronated multifunctional compound as described above.

In some embodiments, the system may include a delivery device that comprises a first reservoir that contains a first composition that comprises the boronated multifunctional compound and the reactive polymer and is buffered to an acidic pH, such as the prepared fluid composition previously described, and a second reservoir that contains second composition, such as the fluid accelerant composition previously described.

In either case, during operation, the first composition and second composition are dispensed from the first and second reservoirs and combined, whereupon the boronated multifunctional compound and the reactive polymer and crosslink with one another to form a crosslinked hydrogel.

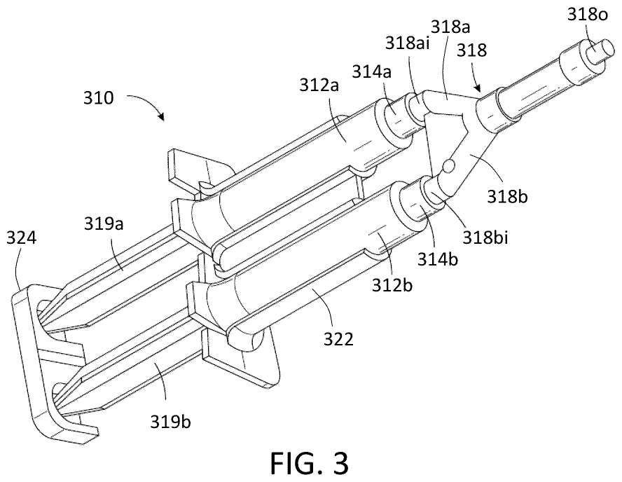

In particular embodiments, and with reference to FIG. 3, the system may include a delivery device 310 that comprises a double-barrel syringe, which includes a first barrel 312a having a first barrel outlet 314a, which first barrel contains a first fluid composition as described above, a first plunger 319a that is movable in the first barrel 312a, a second barrel 312b having a second barrel outlet 314b, which second barrel 312b contains a second fluid composition as described above, and a second plunger 319b that is movable in the second barrel 312b. In some embodiments, the device 310 may further comprise a mixing section 318 (e.g., a Y-connector) having a first mixing section inlet 318ai in fluid communication with the first barrel outlet 314a, a second mixing section inlet 318bi in fluid communication with the second barrel outlet 314b, and a mixing section outlet 318o. Also shown are a syringe holder 322 configured to hold the first and second syringe barrels 312a, 312b, in a fixed relationship and a plunger cap 324 configured to hold the first and second plungers 319a, 319b in a fixed relationship.

In some embodiments, the delivery device may further comprise a needle, sprayer or catheter tube that is configured to receive the first and second fluid compositions from the first and second barrels. For example, a needle, sprayer or catheter tube may be configured to form a fluid connection with an outlet of a mixing section by attaching the needle, sprayer or catheter tube to an outlet of the mixing section, for example, via a suitable fluid connector such as a Luer connector.

As another example, the catheter may be a multi-lumen catheter that comprises a first lumen and a second lumen, a proximal end of the first lumen configured to form a fluid connection with the first barrel outlet and a proximal end of the second lumen configured to form a fluid connection with the second barrel outlet. In some embodiments, the multi-lumen catheter may comprise a mixing section having a first mixing section inlet in fluid communication with a distal end of the first lumen, a second mixing section inlet in fluid communication with a distal end of the second lumen, and a mixing section outlet.

During operation, when the first and second plungers are depressed, the first and second fluid compositions are dispensed from the first and second barrels, whereupon the first and second fluid compositions mix and ultimately crosslink to form a crosslinked hydrogel, which is administered onto or into tissue of a subject. For example, the first and second fluid compositions may pass from the first and second barrels, into the mixing section via first and second mixing section inlets, whereupon the first and second fluid compositions are mixed to form an admixture, which admixture exits the mixing section via the mixing section outlet. In some embodiments, a cannula or catheter tube is attached to the mixing section outlet, allowing the admixture to be administered to a subject after passing through the cannula or catheter tube.

As another example, the first fluid composition may pass from the first barrel outlet into a first lumen of a multi-lumen catheter and the second fluid composition may pass from the second barrel outlet into a second lumen of the multi-lumen catheter. In some embodiments the first and second fluid compositions may pass from the first and second lumen into a mixing section at a distal end of the multi-lumen catheter via first and second mixing section inlets, respectively, whereupon the first and second fluid compositions are mixed in the mixing section to form an admixture, which admixture exits the mixing section via the mixing section outlet.

Regardless of the type of device that is used to mix the first and second fluid compositions or how the first and second fluid compositions are mixed, immediately after an admixture of the first and second fluid compositions is formed, the admixture is initially in a fluid state and can be administered to a subject (e.g., a mammal, particularly, a human) by a variety of techniques including injection and surface application (e.g., by spraying). Alternatively, the first and second fluid compositions may be administered to a subject independently by a variety of techniques, including injection and surface application, and a fluid admixture of the first and second fluid compositions formed in or on the subject. In either approach, a fluid admixture of the first and second fluid compositions is formed, which ultimately crosslinks to form a hydrogel in vivo.

Where formed ex vivo, crosslinked hydrogels may be in any desired form, including a slab, a cylinder, a coating, or a particle. In some embodiments, the crosslinked hydrogel processed into particles of suitable size. Particles may be formed any suitable process, for instance by grinding (including cryogrinding), homogenization, crushing, milling, pounding, pushing through a screen or the like. Sieving or other known techniques can be used to classify and fractionate the particles. Crosslinked hydrogel particles formed using the above and other techniques may vary widely in size, for example, having an average size ranging from 50 to 950 microns.

In addition to a crosslinked hydrogel as described above, crosslinked hydrogel compositions in accordance with the present disclosure may contain additional agents, including therapeutic agents, imaging agents, colorants, tonicity adjusting agents, suspension agents, wetting agents, and pH adjusting agents as described above.

The crosslinked hydrogel compositions of the present disclosure may be sterilized using any suitable method. For example, the compositions may be autoclaved while inside a reservoir, such as a syringe barrel, vial, or ampule by heating the mixture at or to a temperature of about 121° C. Alternatively or additionally, the compositions may be sterilized via sterile filtration and/or by supercritical CO₂, gamma, x-ray or electron beam irradiation. ss

In various embodiments, kits are provided that include one or more delivery devices for delivering the crosslinked hydrogel to a subject. Such systems may include one or more of the following: a syringe barrel, which may or may not contain a crosslinked hydrogel as described herein; a vial, which may or may not contain a crosslinked hydrogel as described here; a needle; a sprayer; a flexible tube (e.g., adapted to fluidly connect the syringe to a needle and/or a sprayer); and an injectable liquid such as water for injection, normal saline or phosphate buffered saline. Whether supplied in a syringe, vial, or other reservoir, the crosslinked hydrogel may be provided in dry form (e.g., powder form) or in a form that is ready for injection, such as an injectable hydrogel form (e.g., a suspension of crosslinked hydrogel particles).

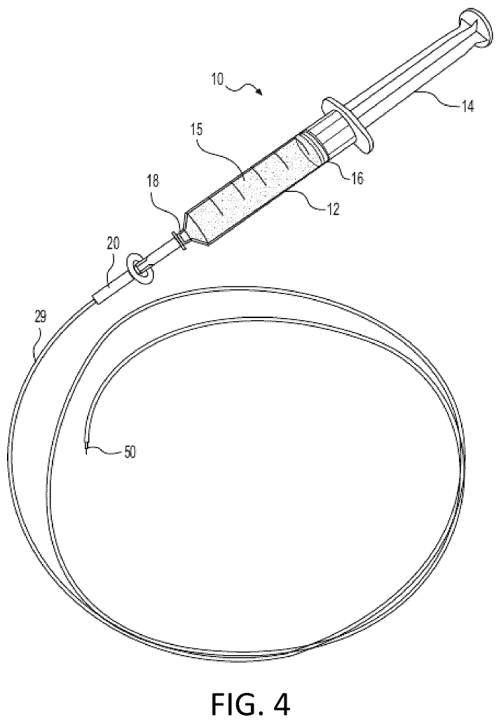

FIG. 4 illustrates a syringe 10 providing a reservoir for a crosslinked hydrogel composition as discussed above. The syringe 10 may comprise a barrel 12, a plunger 14, and one or more stoppers 16. The barrel 12 may include a Luer adapter (or other suitable adapter/connector), e.g., at the distal end 18 of the barrel 12, for attachment to an injection needle 50 via a flexible catheter 29. The proximal end of the catheter 29 may include a suitable connection 20 for receiving the barrel 12. In other examples, the barrel 12 may be directly coupled to the injection needle 50. The syringe barrel 12 may serve as a reservoir, containing a crosslinked hydrogel composition 15 for injection through the needle 50.

In various aspects of the present disclosure, the crosslinked reaction products described herein are useful for boron neutron capture therapy. In a first step, a crosslinked reaction product as described herein is administered to target tissue (e.g., tissues affected by conditions where neutron beam radiation is desired, such as cancer tissue, diseased tissue, etc.). For example, the crosslinked reaction product may be injected into or applied onto target tissue by injecting or applying a crosslinked reaction product that is formed ex vivo as described herein or by injecting or applying first and second fluid compositions that form a crosslinked reaction product in vivo as described herein. During and/or after administration, the crosslinked reaction product may be imaged using a suitable imaging technique. After administration, neutron beam radiation is delivered to the crosslinked reaction product, the target tissue, or both the crosslinked reaction product and the target tissue, leading to the transformation of boron-10 atoms into litihium-7 atoms and alpha particles which can kill or slow the growth of cancer cells, diseased cells, or other target cells in the target tissue. In some embodiments, the neutron beam radiation is applied in a single application. In other embodiments, the neutron beam radiation is applied in multiple applications. It is noted that, in embodiments where the crosslinked reaction product breaks down in vivo, boronated breakdown products are released over time, which may be taken up by the target tissue, enhancing therapeutic efficacy.

Conditions contemplated for treatment by the present disclosure include lesions, surface-based cancers such as bladder cancers, interstitial cystitis, glioblastoma multiforme, meningioma, head and neck cancers, lung cancers, breast cancers, hepatocellular carcinoma, sarcomas, cutaneous malignancies, extramammary Paget's disease, recurrent cancers, pediatric cancers, and metastatic disease.

1. A crosslinked reaction product of (a) a boronated multifunctional compound comprising a plurality of nucleophilic groups and (b) a reactive polymer that comprises a plurality of electrophilic groups that react with the plurality of nucleophilic groups to form covalent linkages.

2. The crosslinked reaction product of claim 1, wherein the boronated multifunctional compound is a boronated polyamino compound that comprises one or more boron atoms and a plurality of amino groups.

3. The crosslinked reaction product of claim 2, wherein the boronated polyamino compound comprises a polyamino moiety that comprises a plurality of amino groups linked to a boronated moiety that comprises one or more boron atoms.

4. The crosslinked reaction product of claim 3, wherein the polyamino moiety is linked to the boronated moiety through a thioester group, an ester group, or an amide group.

5. The crosslinked reaction product of claim 3, wherein the boronated moiety is selected from dioxaborolane-containing moieties and dodecaborate-containing moieties.

6. The crosslinked reaction product of claim 3, wherein the polyamino moiety comprises a plurality of —(CH₂)ₓ—NH₂ groups where x is 1, 2, 3, 4, 5 or 6.

7. The crosslinked reaction product of claim 2, wherein the boronated polyamino compound comprises a poly (amino acid) residue.

8. The crosslinked reaction product of claim 2, wherein the boronated polyamino compound comprises a trilysine residue.

9. The crosslinked reaction product of claim 2, wherein the boronated polyamino compound comprises a residue of a thiol-functionalized dodecaborate-containing compound, a residue of a hydroxy-functionalized dioxaborolane-containing compound, or a residue of an amino-functionalized dioxaborolane-containing compound.

10. The crosslinked reaction product of claim 1, wherein the boronated multifunctional compound comprises an undecahydrododecaborate group or a 4,4,5,5-tetramethyl-1,3,2-dioxaborolan-2-yl group.

11. The crosslinked reaction product of claim 1, wherein the reactive polymer is a reactive multi-arm polymer that comprises a plurality of hydrophilic polymer arms having electrophilic end groups.

12. The crosslinked reaction product of claim 11, wherein the electrophilic end groups are cyclic imide ester groups.

13. The crosslinked reaction product of claim 1, wherein the reactive polymer is a reactive multi-arm polymer that comprises three or more polymer arms linked to a core region, each of the polymer arms comprising a hydrophilic polymer segment and an electrophilic end group.

14. The crosslinked reaction product of claim 13, wherein each of the polymer arms comprises a hydrolysable ester group disposed between the hydrophilic polymer segment and the electrophilic end group.

15. The crosslinked reaction product of claim 13, wherein the hydrophilic polymer segment is selected from polyalkylene oxide segments, polyester segments, polyoxazoline segments, polydioxanone segments, and polypeptide segments.

16. The crosslinked reaction product of claim 13, wherein the core region comprises a polyol residue.

17. The crosslinked reaction product of claim 1, wherein the crosslinked reaction product is a hydrogel.

18. A system for forming a crosslinked reaction product comprising (a) a first composition in a first container, the first composition comprising a boronated multifunctional compound comprising a plurality of nucleophilic groups and a reactive polymer that comprises a plurality of electrophilic groups that react with the plurality of nucleophilic groups to form covalent linkages, (b) a second composition that comprises an acidic buffer in a second container, and (c) a third composition that comprises a basic buffer in a third container.

19. The system of claim 18, wherein the first container, the second container and the third container are independently selected from vials and syringe barrels.

20. A method of treatment comprising (a) applying, injecting, or forming a crosslinked reaction product on or in target tissue of a subject, the crosslinked reaction product comprising (i) a boronated multifunctional compound comprising a plurality of nucleophilic groups and (ii) a reactive polymer that comprises a plurality of electrophilic groups that react with the plurality of nucleophilic groups to form covalent linkages, and (b) delivering neutron beam radiation to the target tissue, the crosslinked reaction product, or both.
